(19) United States
(12) Patent Application Publication
(10) Pub. No.: US 2015/0038355 A1
(43) Pub. Date: Feb. 5, 2015

(54) MULTIPLEX BLOCKER BEADS FOR IMMUNOASSAYS

(71) Applicant: Bio-Rad Laboratories, Inc., Hercules, CA (US)

(72) Inventors: Woei Tan, Hercules, CA (US); Deepa Jethwaney, Dublin, CA (US); Vinita Gupta, Danville, CA (US); Qian-Shu Wang, San Ramon, CA (US); Doris Yeung, Hercules, CA (US)

(21) Appl. No.: 14/341,594

(22) Filed: Jul. 25, 2014

Related U.S. Application Data
(60) Provisional application No. 61/860,009, filed on Jul. 30, 2013.

(51) Int. Cl. G01N 33/543

(52) U.S. Cl. CPC G01N 33/54393
USPC 506/9; 506/15; 506/18; 506/20

(57) ABSTRACT

Provided are methods and compositions for immunoassay with improved specificity. The presently disclosed bead-based blocking agents reduce the interference associated with the samples and reagents of such assays.

### CROSS-REFERENCES TO RELATED APPLICATIONS

This application claims priority to U.S. Provisional Application No. 61/860,009, filed on Jul. 30, 2013, which is hereby incorporated by reference in its entirety for all purposes.

### BACKGROUND OF THE INVENTION

Serological immunoassays (e.g., affinity assays) can provide sensitive and specific means for the quantitation of analytes, e.g., biomolecules in biological samples. However, the assays are susceptible to non-specific reactivity of the assay reagents, which cause interference in the measurements of the analytes. Serum and plasma samples can also cause matrix effects or non-specific reactivity such as high background. Certain proteins present in the samples, such as heterophile antibodies, can also increase the non-specific signal or results in false-positives.

Solid phase immunoassays (e.g., ELISA or microparticle based immunoassays) involve the immobilization of biomolecules to the surface of the solid phase (e.g., well or microparticle) via passive or covalent interactions. Non-specific binding of interfering or competing biomolecules in the sample can attach to the assay reagents or to unoccupied sites on the solid phase, thereby hindering the accurate detection of the target molecule.

### BRIEF SUMMARY OF THE INVENTION

The presently disclosed blocking agents can be used to separate interfering molecules in a sample, resulting in increased sensitivity of an immunoassay.

A method is provided for blocking an interfering molecule in an affinity assay mixture, wherein the mixture comprises a sample. In some aspects, the method comprises (a) contacting a first blocking agent to the sample, wherein the first blocking agent comprises a bead linked to a first binding agent, thereby forming a non-specific binding complex between the first binding agent and one or more molecules in the sample that would otherwise interfere with forming an affinity complex between an affinity agent and a target molecule, if present, in the sample; and (b) contacting a second blocking agent to the sample, wherein the second blocking agent comprises a bead linked to a second binding agent that is different from the first binding agent, thereby forming a non-specific binding complex between the second binding agent and one or more molecules in the sample that would otherwise interfere with forming an affinity complex between the affinity agent and the target molecule, if present, in the sample; thereby blocking the interfering molecule in the assay mixture.

In some embodiments, the first binding agent and the second binding agent bind to different interfering molecules. In other embodiments, the first binding agent and the second binding agent bind to the same interfering molecule. In some embodiments, the binding agent is selected from the group consisting of BSA, protein L, collagen, PEG4000/6000, animal serum, a murine based IgG aggregate, and an antibody derived from goat, mouse, rabbit or sheep that recognizes a HAGA, HAMA, HARA, HASA, or rheumatoid factor.

In some embodiments, the first blocking agent comprises more than one binding agent. In some embodiments, the second blocking agent comprises more than one binding agent.

In some embodiments, the first blocking agent and the second blocking agent are at a substantially equal ratio. In other embodiments, the first blocking agent is at a ratio of at least about 2 times or more than the second blocking agent.

In some embodiments, the bead of the blocking agent is a non-magnetic bead. In other embodiments, the bead of the blocking agent is a bead that is responsive to a magnetic field.

In some embodiments, the bead of the blocking agent is a fluorescent bead. In other embodiments, the bead of the blocking agent is a non-fluorescent bead. In some instance, the bead does not generate a fluorescent signal.

In some embodiments, the method further comprises removing the non-specific binding complex from the other components of the assay mixture. In some aspects, the method of removing the non-specific binding complex comprises applying a magnetic field to the assay mixture. In some aspects, method of removing the non-specific binding complex comprises centrifuging the assay mixture.

In some embodiments, the assay mixture comprises a plurality of different fluorescent beads and the bead of the blocking agent comprises a fluorescent dye distinguishable from other dyes in the plurality. In some instances, the fluorescent beads are detected and quantitated.

In some embodiments, the method further comprises contacting the affinity agent to the sample; and detecting the presence or amount of the affinity complex. In some embodiments, the method further comprises contacting the affinity agent to the sample; separating the affinity complex from other components of the assay mixture; and detecting the presence or amount of the affinity complex mixture. In some aspects, separating the affinity complex comprises applying a magnetic field to the assay mixture.

In some embodiments, the method further comprises before contacting the affinity agent to the sample, contacting the blocking agent to an affinity agent solution, thereby forming a non-specific binding complex between the binding agent and one or more interfering molecules in the affinity agent solution; and separating the non-specific binding complex from other components of the affinity agent solution to generate the affinity agent.

In some embodiments, the affinity agent comprises an antibody or a fragment thereof. In some embodiments, the affinity agent further comprises a bead. In one aspect, the bead is responsive to a magnetic field.

In some instances, the assay mixture comprises a plurality of different fluorescent beads and the bead of the affinity agent comprises a fluorescent dye distinguishable from other dyes in the plurality.

In some embodiments, the assay mixture comprises more than one affinity agent. In some embodiments, the assay mixture comprises more than two blocking agents.

In another aspect, provided herein is a plurality of blocking agents comprising a first bead linked to a first binding agent and a second bead linked to a second binding agent that is different that the first binding agent, wherein the binding agent is selected from the group consisting of BSA, protein L, collagen, PEG4000/6000, animal serum, a murine based IgG aggregate, and an antibody derived from goat, mouse, rabbit or sheep that recognizes a HAGA, HAMA, HARA, HASA, or rheumatoid factor.

In some embodiments, the bead comprises a fluorescent dye. In other embodiments, the bead does not generate a fluorescent signal. In some embodiments, the bead is a non-magnetic bead. In other embodiments, the bead is responsive to a magnetic field.

In yet another aspect, provided herein is a kit comprising a plurality of blocking agents and an affinity agent specific for a target molecule, wherein the plurality of blocking agents comprises a first blocking agent comprising a bead linked to a first binding agent that forms a non-specific binding complex to one or more interfering molecules in a sample and a second blocking agent comprising a bead linked to a second binding agent that is different than the first binding agent and forms non-specific binding complex to one or more interfering molecules in a sample.

In some embodiments, the bead is a non-magnetic bead. In some embodiments, the bead is responsive to a magnetic field.

In some embodiments, the binding agent is selected from the group consisting of BSA, protein L, collagen, PEG4000/6000, animal serum, a murine based IgG aggregate, and an antibody derived from goat, mouse, rabbit or sheep that recognizes a HAGA, HAMA, HARA, HASA, or a rheumatoid factor. In some instances, the bead of the first blocking agent is linked to at least two different binding agents. In some instances, the bead of the second blocking agent is linked to at least two different binding agents.

In some embodiments, the bead comprises a fluorescent dye that is distinguishable from other components of the kit. In some embodiments, the bead does not generate a fluorescent signal.

In some embodiments, the affinity agent comprises an antibody or a fragment thereof. In some embodiments, the affinity agent further comprises bead. In some instances, the bead is responsive to a magnetic field. In some instances, the bead comprises a fluorescent dye that is distinguishable from other components of the kit.

In some embodiments, the kit comprises more than two blocking agents. In some embodiments, the kit comprises more than one affinity agent.

In another aspect, a method is provided for blocking an interfering molecule in an affinity assay mixture, the method comprises contacting a blocking agent to a sample, wherein the blocking agent comprises a bead linked to at least two different binding agents, thereby forming an non-specific binding complex between the binding agent and one or more molecule in the sample that would otherwise interfere with forming an affinity complex between an affinity agent and a target molecule, if present, in the sample, thereby blocking the interfering molecule in the assay mixture.

In some embodiments, the bead of the blocking agent is a non-magnetic bead. In other embodiments, the bead of the blocking agent is a bead that is responsive to a magnetic field.

In some embodiments, at least one binding agent is selected from the group consisting of BSA, protein L, collagen, PEG4000/6000, animal serum, a murine based IgG aggregate, and an antibody derived from goat, mouse, rabbit or sheep that recognizes a HAGA, HAMA, HARA, HASA, and rheumatoid factor. In other embodiments, at least two binding agents are selected from the group consisting of BSA, protein L, collagen, PEG4000/6000, animal serum, a murine based IgG aggregate, and an antibody derived from goat, mouse, rabbit or sheep that recognizes a HAGA, HAMA, HARA, HASA, or rheumatoid factor.

In some embodiments, the method further comprises removing the non-specific binding complex from the other components of the assay mixture. In some instances, the step of removing the non-specific complex comprises applying a magnetic field to the assay mixture.

In some embodiments, the assay mixture comprises a plurality of different fluorescent beads and the bead of the blocking agent comprises a fluorescent dye distinguishable from other dyes in the plurality. In other embodiments, the bead of the blocking agent does not generate a fluorescent signal.

In some embodiments, the fluorescent beads of the assay mixture are detected and quantitated.

In one embodiment, the method further comprises contacting the affinity agent to the sample; separating the affinity complex from the other components of the assay mixture; and detecting the presence or amount of the affinity complex. In some instances, the step of separating the affinity complex comprises applying a magnetic field to the assay mixture.

In other embodiments, the method further comprises before contacting the affinity agent to the sample, contacting the blocking agent to the affinity agent solution, thereby forming a non-specific binding complex between the binding agent and one or more interfering molecules in the affinity agent solution; and separating the non-specific binding complex from other components of the affinity agent solution to generate the affinity agent. The affinity agent can be used in the method described herein or in other single plex or multiplex affinity assays known to those in the art, such as an ELISA.

In some embodiments, the affinity agent comprises an antibody or a fragment thereof. In other embodiments, the affinity agent further comprises a bead. In some instances, the bead of the affinity agent is responsive to a magnetic field. In some embodiments, the assay mixture comprises a plurality of different fluorescent beads and the bead of the affinity agent comprises a fluorescent dye distinguishable from other dyes in the plurality.

In some embodiments, the assay mixture comprises more than one affinity agent. In some embodiments, the assay mixture comprises more than one blocking agent.

In another aspect, provided herein is a blocking agent comprising a bead linked to at least two different binding agents, wherein at least one binding agent is selected from the group consisting of BSA, protein L, collagen, PEG4000/6000, animal serum, a murine based IgG aggregate, and an antibody derived from goat, mouse, rabbit or sheep that recognizes a HAGA, HAMA, HARA, HASA, or rheumatoid factor. In another aspect, provided herein is a blocking agent comprising a bead linked to at least two different binding agents, wherein at least one binding agent is selected from the group consisting of BSA, protein L, collagen, PEG4000/6000, animal serum, a murine based IgG aggregate, and an antibody derived from goat, mouse, rabbit or sheep that recognizes a HAGA, HAMA, HARA, HASA, or rheumatoid factor.

In some embodiments, the bead comprises a fluorescent dye. In some embodiments, the bead does not generate a fluorescent signal. In some embodiments, the bead is a non-magnetic bead. In some embodiments, the bead is responsive to a magnetic field.

In another aspect, provided herein is a kit comprising a blocking agent and an affinity agent specific for a target molecule, wherein the blocking agent comprises a bead linked to at least two different binding agents, and forms a non-specific binding complex to one or more interfering molecules in a sample.

In some embodiments, the bead of the blocking agent is responsive to a magnetic field. At least one (or at least two) binding agent can be selected from the group consisting of BSA, protein L, collagen, PEG4000/6000, animal serum, a murine based IgG aggregate, and an antibody derived from goat, mouse, rabbit or sheep that recognizes a HAGA, HAMA, HARA, HASA, or rheumatoid factor.

In some embodiments, the bead of the blocking agent comprises a fluorescent dye that is distinguishable from other components of the kit. In some embodiments, the bead does not generate a fluorescent signal.

In some embodiments, the affinity agent of the kit further comprises a bead. In some instances, the bead of the affinity agent is responsive to a magnetic field. In some instances, the bead of the affinity agent comprises a fluorescent dye that is distinguishable from other components of the kit.

In some embodiments, the kit comprises more than one blocking agent. In some embodiments, the kit comprises more than one affinity agent.

### BRIEF DESCRIPTION OF THE DRAWINGS

FIG. 1 illustrates an exemplary embodiment of a method of using blocking reagents in a multiplex immunoassay.

### DETAILED DESCRIPTION OF THE INVENTION

Interfering molecules including, but not limited to, polyreactive antibodies and autoantibodies that are present in clinical samples, such as serum prevent the accurate measurement of target analytes, such as proteins by immunoassays. In particular, these molecules interfere with the binding reaction between the target analyte and its affinity agent (e.g., affinity antibody) in an immunoassay.

The inventors have developed a method of using one or more blocking agents in a multiplex solid phase immunoassay to reduce the effect of non-specific binding of an interfering molecule to an affinity agent and to allow the specific binding of the target analyte and the affinity agent. The blocking agent(s) used in a particular assay is selected, for example, based on the type of interfering molecule(s) found or expected to be present in the biological sample.

The blocking agent can be a solid-phase bead attached to at least one (and in some embodiments at least two different) binding agent that can form a complex with one or more interfering molecules in a sample, thereby sequestering the interfering molecule from other components of the assay such as the sample and/or the affinity agent. Furthermore, the blocking agent itself does not affect antibody binding of the target analyte. The blocking agent can be exposed to the sample alone, the affinity agent alone, or the assay mixture containing the sample and the affinity agent.

The specificity of the blocking agent for a particular interfering molecule is dependent on its binding agent. A sample can contain multiple types of interfering molecules, and therefore, more than one type of binding agent or blocking agent can be used. The selection of the blocking agent will depend on the sample and the assay reagents, and thus can be determined empirically.

The blocking agents can be used in any affinity assay, e.g. an immunoassay. In standard immunoassays, the blocking agent can be a non-magnetic, and optionally, fluorescent bead. For magnetic bead-based assays, the blocking agent can be a superparamagnetic, and optionally, fluorescent bead.

Provided herein are methods, compositions and kits for bead-based blocking agents for affinity immunoassays.

### I. DEFINITIONS

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, N.Y. 1989). The term “a” or “an” is intended to mean “one or more.” The term “comprise” and variations thereof such as “comprises” and “comprising,” when preceding the recitation of a step or an element, are intended to mean that the addition of further steps or elements is optional and not excluded. Any methods, devices and materials similar or equivalent to those described herein can be used in the practice of this invention. The following definitions are provided to facilitate understanding of certain terms used frequently herein and are not meant to limit the scope of the present disclosure.

The term “affinity assay mixture” or “assay mixture” refers to the reaction mixture of an affinity-based assay wherein a target molecule is detected in sample by specific binding of the target molecule to a binding partner such as, but not limited to, an antibody or a fragment thereof. An assay mixture can include a sample, blocking agent, affinity agent, binding agent, buffer, washing buffer, or combinations thereof.

The term “blocking agent” refers to a solid phase reagent that directly binds an interfering or competing molecule present in a sample or in reagents of an immunoassay. As described herein, a blocking agent comprises a solid phase bead and one or more binding agent, wherein the binding agent is coupled (e.g., conjugated, linked or bound) to the bead.

As used herein, the term “bead” includes a particle, microbead, microparticle, microsphere, nanobead, nanoparticle, nanosphere or the like. In various embodiments, commerically available beads or other particles, e.g., Miltenyi Particles, Miltenyi Biotec, Germany; Sepharose beads, Pharmacia Fine Chemicals, Sweden; DYNABEADS™, Dynal Inc., Oslo, Norway; PuraBead™, ProMetic Biosciences, Rockville, Md.; magnetic beads from Immunicon, Huntingdon Valley, Pa., microspheres from Bangs Laboratories, Inc., Fishers, Ind., are useful.

As used herein, the term “paramagnetism” refers to magnetism that occurs only the presence of an externally applied magnetic field. The term “superparamagnetic” in reference to a bead, as defined above, is defined as not retaining any significant amount of magnetization in the absence of an extenally applied magnetic field, and thus does not form aggregates.

The term “reactive group” refers to a chemical moiety on the compound that is capable of chemically reacting with a functional group on a separate, different compound to form a covalent linkage.

The term “binding agent” refers to a reagent that binds an interfering molecule(s) present in a sample or in reagents of an immunoassay. It can also fill unoccupied sites on a solid phase substrates, such as a bead, well, membrane, etc. Examples include, but are not limited to, bovine serum albumin, milk solids, non-protein-based reagent, protein-based reagent, a surfactant (e.g., Tween 20, Triton X-100, CHAPS), casein and derivatives thereof, gelatin (e.g., fish gelatin), collagen, Protein A, Protein G, Protein L, polymers (e.g., polyethylene glycol (PEG), polyvinyl alcohol, polyvinylpryyolidone), animal serum, non-animal serum, immunoglobulins or immunoglobulin aggregates, heterophilic antibody, commercial blocking substances, and natural or synthetic peptides.

The term “affinity agent” refers to a molecule that specifically binds to a target molecule. Non-limiting examples of an affinity agent include an antibody, antibody fragments (e.g., Fab, F(ab′)2, Fv, scFv, Fd, scFv-Fc, ScFv-CH, scFab, scFv-zipper), aptamer, ligand, enzyme, antigen and polypeptide.

The term “target molecule” or “target analyte” is used herein to refer to a molecule, compound, or complex that is recognized by an affinity agent, i.e., can be specifically bound by the antibody or a fragment thereof. The term can refer to any molecule that can be specifically recognized by an antibody or fragment thereof, e.g., a polypeptide, polynucleotide, carbohydrate, lipid, chemical moiety, or combinations thereof (e.g., phosphorylated or glycosylated polypeptides, chromatin moieties, etc.). One of skill will understand that the term does not indicate that the molecule is immunogenic in every context, but simply indicates that it can be targeted by an antibody.

The term “interfering molecule” is used herein to refer to a molecule, compound, or complex that leads to non-specific reactivity, high background, and/or false-positives in an immunoassay. An interfering molecule can decrease the specificity of an immunoassay. An interfering molecule is not the target analyte (molecule) in an affinity assay (e.g., immunoassay or a multiplex immunoassay).

As used herein, the term “rheumatoid factor” or “RF” refers an autoantibody that typically binds to the Fc portion of an antibody (e.g., the IgG class), and is generally cross-reactive to various species. Rheumatoid factors can interfere with immunological assays for specific analytes. For example, in a sandwich assay, RF can bridge between the affinity antibody and the detection antibody producing an artifically elevated signal for the target molecule. In a competitive assay, RF can block the binding of a labeled target molecule to the affinity antibody and generate falsely low signals.

As used here in, the term “heterophilic antibody” refers to an antibody with multispecificity by having multiple binding sites or by having a single binding site that can recognize a number of antigens with similar structures. Heterophile antibodies are present in 5-40% of normal blood samples. These interfering antibodies react to poorly defined antigens and generally show weak avidity and are multispecies specific. For instance, a heterophile antibody in human serum can be reactive to goat, mouse, and rat proteins.

The term “antibody” refers to a polypeptide structure, e.g., an immunoglobulin, conjugate, or fragment thereof that retains antigen binding activity. The term includes but is not limited to polyclonal or monoclonal antibodies of the isotype classes IgA, IgD, IgE, IgG, and IgM, derived from human or other mammalian cells, including natural or genetically modified forms such as humanized, human, single-chain, chimeric, synthetic, recombinant, hybrid, mutated, grafted, and in vitro generated antibodies. The term encompasses conjugates, including but not limited to fusion proteins containing an immunoglobulin moiety (e.g., chimeric or bispecific antibodies or scFv's), and fragments, such as Fab, F(ab′)2, Fv, scFv, Fd, dAb and other compositions.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. The variable region contains the antigen-binding region of the antibody (or its functional equivalent) and is most critical in specificity and affinity of binding. See Paul, Fundamental Immunology (2003).

Antibodies can exist as intact immunoglobulins or as any of a number of well-characterized fragments that include specific antigen-binding activity. Such fragments can be produced by digestion with various peptidases. Pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies or those identified using phage display libraries (see, e.g., McCafferty et al., Nature 348:552-554 (1990)).

As used herein, the term “Fv” refers to a monovalent or bi-valent variable region fragment, and can encompass only the variable regions (e.g., V_{L} and/or V_{H}), as well as longer fragments, e.g., an Fab, Fab′ or F(ab′)2, which also includes C_{L} and/or C_{H}1. Unless otherwise specified, the term “Fc” refers to a heavy chain monomer or dimer comprising C_{H}1 and C_{H}2 regions.

A single chain Fv (scFv) refers to a polypeptide comprising a V_{L} and V_{H} joined by a linker, e.g., a peptide linker. ScFvs can also be used to form tandem (or di-valent) scFvs or diabodies. Production and properties of tandem scFvs and diabodies are described, e.g., in Asano et al. (2011) J Biol. Chem. 286:1812; Kenanova et al. (2010) Prot Eng Design Sel 23:789; Asano et al. (2008) Prot Eng Design Sel 21:597.

The terms “specific for,” “specifically binds,” and like terms refer to the binding of a molecule (e.g., antibody or antibody fragment) to a target (antigen, epitope, antibody target, etc.) with at least 2-fold greater affinity than non-target compounds, e.g., at least 4-fold, 5-fold, 6-fold, 7-fold, 8-fold, 9-fold, 10-fold, 20-fold, 25-fold, 50-fold, or 100-fold greater affinity. For example, an antibody that specifically binds, or is specific for, a primary antibody will typically bind the primary antibody with at least a 2-fold greater affinity than a non-primary antibody target (e.g., an antibody from a different species or of a different isotype, or a non-antibody target).

The term “binds” with respect to an antibody target (e.g., antigen, analyte, immune complex), typically indicates that an antibody binds a majority of the antibody targets in a pure population (assuming appropriate molar ratios). For example, an antibody that binds a given antibody target typically binds to at least ⅔ of the antibody targets in a solution (e.g., 75, 80, 85, 90, 91, 92, 93, 94, 95, 96, 97, 98, 99, or 100%). One of skill will recognize that some variability will arise depending on the method and/or threshold of determining binding.

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

### II. BLOCKING AGENTS

A. Binding Agent

The presently described blocking agents can comprise one or in some aspects, at least two different binding agents. The binding agent can bind to one or more interfering molecules in solution with an affinity agent, or in a sample. In addition, the binding agent does not substantially bind to the intended target analyte to which the affinity agent specifically binds. Furthermore, the binding agent does not substantially bind to the target analytes of a multiplex affinity assay.

Any reagent that can bind an interfering molecule present in an immunoassay sample can be used as a binding agent. Exemplary reagents are described in detail in, e.g., Crowther, J R., 1995, Methods Mol. Biol., 42:1-223; Davies, C., 1994. “Concepts.” In the Immunoassay Handbook, D. Wild, ed., Stockton Press, New York, p. 83-115, and Hornbeck, P., 2001, “Enzyme-Linked Immunosorbent Assays.” In Current Protocols in Immunology, Unit 2.1, ed., R. Coico, John Wiley & Sons, Hoboken, N.J. Examples of binding agents include, but are not limited to, BSA, protein L, collagen, PEG4000/6000, whole normal animal serum (e.g. mouse serum, rat serum, goat serum, rabbit serum, sheep serum), an animal based IgG aggregate (e.g., mouse IgG, rat IgG, rabbit IgG, goat IgG, sheep IgG), and an antibody derived from goat, mouse, rabbit or sheep that recognizes a HAGA, HAMA, HARA, HASA, or rheumatoid factor. Examples of commercially available binding agents include Superchemiblock heterophile blocking agent (Millipore, Billerica, Mass.), immunoglobulin-inhibiting reagent (IIR; Bioreclamation, Inc., Westbury, N.Y.), heterophile blocking tubes (Scantibodies Laboratory, Santee, Calif.), and StabliGuard immunoassay stabilizer (SurModics, Inc., Eden Prairie, Minn.).

In some embodiments, the binding agent can be an antibody (e.g., IgG, IgG, IgM, IgE or IgD), e.g., of animal (e.g., mouse, rabbit, sheep, goat, donkey, etc.) origin. Such antibodies can, for example, specifically bind and neutralize a heterophilic antibody, a rheumatoid factor, or other interfering molecule. For instance, the attachment of the immunoglobulin to a heterophilic antibody prevents the heterophilic antibody from binding (capturing) an antibody that is specific for the target analyte or a detection antibody. The binding agent can be antibody that cannot bind to the target analyte or the affinity antibody that is specific for (e.g., can specifically bind to) the target analyte.

In some embodiments, the binding agent binds to one or more interfering molecule. In particular embodiments, more than one binding agent (e.g., a first binding agent and a second binding agent) can bind the same interfering molecule.

In some embodiments, the blocking agent comprises a first binding agent and a second binding agent. In some instances, the first and second binding agents bind the same interfering molecule. In some instances, the first and second binding agents do not bind the same interfering molecule. In some embodiments, the blocking agent comprises a plurality of binding agents, e.g., 2, 3, 4, 5, 6, 7, 8, 9, 10 or more binding agents. In some embodiments, the binding agents bind to a plurality of different types of interfering molecules, e.g., 2, 3, 4, 5, 6, 7, 8, 9, 10 or more interfering molecules.

One or more binding agent can be used in an affinity assay such as a multiplex affinity assay to reduce the effects of interfering molecules. In some embodiments, the binding agents are utilized in about a one-to-one ratio. For instance, the first binding agent and the second binding agent are present in a substantially equal amount (e.g., concentration). If more than two binding agents are present, all binding agents can be in a substantially equal amount. In other embodiments, the binding agents are used in differing amounts (e.g., concentrations). In some embodiments, the first binding agent is at an amount that is about 2, 3, 4, 5, 6, 7, 8, 9, 10, 11, 12, 13, 14, 15, 16, 17, 18, 19, 20, or more time greater than the second binding agent.

The amount of binding agent(s) for the blocking agent used in the method provided herein can be determined empirically. For example, titration experiments with the blocking agent can be performed to establish the optimal amount of binding agent(s) needed for a particular blocking agent in a particular affinity assay or with a specific sample type.

B. Bead

The presently described blocking agent comprises a bead. In some embodiments, the bead size ranges from about 1 μm to about 100 μm or more, e.g., 1, 5, 10, 15, 20, 25, 30, 35, 40, 45, 50, 55, 60, 65, 70, 75, 80, 85, 90, 95, 100 μm or more in diameter. In other embodiments, the bead size ranges from about 1 nm to about 1000 nm or more, e.g., 1, 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 nm or more in diameter. The beads can be of substantially uniform size.

In some embodiments, the bead is a nonmagnetic bead. The bead can be made of polymer material, such as, but not limited to polystyrene, brominated polystyrene, polyacrylic acid, polyacrylonitrile, polyamide, polyacrylamide, polyacrolein, polybutadiene, polycaprolactone, polycarbonate, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze, polysulfone, or combinations thereof. The bead can also be made of carbohydrate (e.g., carboxymethyl cellulose, hydroxyethyl cellulose), agar, gel, proteinaceous polymer, polypeptide, eukaryotic and prokaryotic cells, viruses, lipid, metal, resin, latex, rubber, silicone, (e.g., polydimethyldiphenyl siloxane), glass, ceramic, charcoal, kaolinite, bentonite, and the like.

In some embodiments, the bead is responsive to a magnetic field. In some instances, the bead is magnetic, superparamagnetic, paramagnetic, or ferromagnetic. These beads can comprise a coating material, e.g., a material that is attracted to another material in a magnetic field, such as iron oxide (e.g., magnetite, maghemite), magnesium, molybdenum, lithium and tantalum.

C. Reactive Groups

A reactive group can be an electron pair donor or acceptor that can form a chemical bond when reacted to a corresponding functional group. The reactive group can be on the binding agent or on the bead of the blocking agent. For instance, the conjugation reaction between the reactive group on the bead and the binding agent to be conjugated can results in one or more atoms of the reactive group to be incorporated into a new linkage attaching the binding agent to the bead.

Examples of the electron pair acceptor group include, but are not limited to, an activated ester (including an N-hydroxysuccinimide ester or a tetrafluorophenyl ester), an acrylamide, an acyl azide, an acyl halide, an acyl nitrile, an aldehyde, a ketone, an alkyl halide, an alkyl sulfonate, an alkyl thiosulfonate, an anhydride, an aryl halide, an azide, an aziridine, a boronate, a carbodiimide, a diazoalkane, a diene, an epoxide, a haloacetamide, a haloplatinate, or a halotriazine.

Examples of the electron pair donor group include, but are not limited to, a thiol, an amine, an alcohol, a hydrazine, a hydroxylamine, a carboxylic acid, a cycloalkyne, an ester-substituted triaryl phosphine, a glycol, a benzoquinone, or a heterocycle.

Examples of the covalent linkage include, but are not limited to, a carboxamide, thioether, ester, imine, hydrazine, oxime, alkyl amine, ether, disulfide, phenyl thioether, aryl amine, 1,2,3-triazole, amide, boronate ester, N-acrylurea, cyclohexene, and aminotriazine bond. The linkage can be, but is not limited to, an ether, thioether, carboxamide, sulfonamide, urea, urethane or hydrazine moiety.

The covalent linkage binds the reactive group of the binding agent (or bead) to the functional group on the bead (or binding agent), either directly (e.g., a single bond) or with a combination of stable chemical bonds, such as, for example, single, double, triple or aromatic carbon-carbon bonds, as well as carbon-nitrogen bonds, nitrogen-nitrogen bonds, carbon-oxygen bonds, carbon-sulfur bonds, phosphorus-oxygen bonds and phosphorus-nitrogen bonds.

Selection of the reactive group used to attach the binding agent to the bead depends on the functional group on the bead and the type and/or length of covalent linkage desired. The types of functional groups typically present on a bead include, but are not limited to, amines, amides, thiols, alcohols, phenols, aldehydes, ketones, phosphates, imidazoles, hydrazines, hydroxylamines, disubstituted amines, halides, epoxides, carboxylate esters, sulfonate esters, purines, pyrimidines, carboxylic acids, olefinic bonds, or a combination of these groups. In some embodiments, the bead of the blocking agent is a carboxyl-modified bead, amino-modified bead, hydroxyl-modified bead, hydrazide-modified bead, or chloromethyl-modified bead.

A bead (e.g., nonmagnetic or responsive to a magnetic field) may have more than one functional group, and can be conjugated to more than one binding agent through different covalent linkages. For instance, a first binding agent can be coupled (e.g., attached, linked) to the bead via a first reactive group and a second binding agent can be coupled to the same bead through a second, different reactive group.

The reactive group of the binding agent can be, but is not limited to, an amine, a thiol, an alcohol, an aldehyde or a ketone, an acrylamide, a reactive amine (including a cadaverine or ethylenediamine), an activated ester of a carboxylic acid (typically a succinimidyl ester of a carboxylic acid), an acyl azide, an acylnitrile, an aldehyde, an alkyl halide, an anhydride, an aniline, an aryl halide, an azide, an aziridine, an aliphatic amine, a boronate, a carboxylic acid, a diazoalkane, a haloacetamide, a halotriazine, a hydrazine (including hydrazides), an imido ester, an isocyanate, an isothiocyanate, a maleimide, a phosphoramidite, a perfluorobenzamido, an azidoperfluorobenamido group, a psoralen, a reactive platinum complex (including a haloplatinate or a platiunum nitrate), a sulfonyl halide, a thiol group, or a variant thereof.

Further examples of reactive groups and covalent linkages between beads and binding agents are found in, e.g., S. S. Wong, “Chemistry of Protein Conjugation and Cross-Linking,” CRC Press (1991) and G. T. Hermanson, “Bioconjugate Techniques,” Academic Press (1995). For instance, proteins, such as BSA, and antibodies can be coupled to a bead by conjugating the free amines of lysine residues and/or the N-terminal amines of the proteins to the carboxyl groups on the bead.

D. Fluorescent Dyes

The blocking agent provided herein can comprise a fluorescent dye with light emission at a wavelength in the ultraviolet or visible light spectra range. Non-limiting examples of fluorescent dyes include xanthenes (fluoresceins, rhodamines, 6-carboxyfluorescein, 6-carboxy-4′,5′-dichloro-2′,7′-dimethoxyfluorescein, N,N,N′,N′-tetramethyl-6-carboxyrhodamine, 6-carboxy-X-rhodamine, 5-carboxyrhodamine-6G, 5-carboxyrhodamine-6G, tetramethylrhodamine, Rhodamine Green, and Rhodamine Red), cyanines, cyanine succinimidyl esters (sulfoindocyanine succinimidyl esters, (carboxyalkyl)cyanines succinimidyl esters, BODIPY succinimidyl esters), coumarins (umbelliferone), benzimides (Hoeschst 33258), phenanthridines (Texas Read), ethidium dyes, acridine dyes, carbazole dyes, phenoxazine dyes, porphyrin dyes, and quinoline dyes. Examples of fluorescent dyes also include 4-acetamido-4′-isothiocyanatostilbene-2,2′ disulfonic acid, acridine, acridine isothiocyanate, 5-(2′-aminoethyl)aminonaphthalene-1-sulfonic acid (EDANS),4-amino-N-[3-vinylsulfonyl)phenyl]naphthalimide-3,5 disulfonate, N-(4-anilino-1-naphthyl)maleimide, anthranilamide, BODIPY, Brilliant Yellow, coumarin, 7-amino-4-methylcoumarin (AMC, Coumarin 120), 7-amino-4-trifluoromethylcoumarin (Coumaran 151), cyanine dyes, cyanosine, 4′,6-diaminidino-2-phenylindole (DAPI), 5′,5″-dibromopyrogallol-sulfonaphthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansylchloride), 4-(4′-dimethylaminophenylazo)benzoic acid (DABCYL), 4-dimethylaminophenylazophenyl-4′-isothiocyanate (DABITC), eosin, eosin isothiocyanate, erythrosin B, erythrosin isothiocyanate, ethidium, 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, Fluorescamine, IR144, IR1446, Malachite Green isothiocyanate, 4-methylumbelliferone, ortho cresolphthalein, Pararosaniline, Phenol Red, B-phycoerythrin, o-phthaldialdehyde, pyrene, pyrene butyrate, succinimidyl 1-pyrene butyrate, quantum dots, Reactive Red 4 (Cibacron™ Brilliant Red 3B-A), 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, tetramethyl rhodamine isothiocyanate (TRITC), riboflavin, rosolic acid, lanthanide chelate derivatives, and derivatives thereof. One skilled in the art will recognize that the fluorescent dye can be any dye that is capable of emitting a fluorescent detectable signal.

The fluorescent beads can be prepared by organic synthesis methods by means well recognized in the art (see, e.g., Haugland, MOLECULAR PROBES HANDBOOK, supra, (2002)). Fluorescent dye or fluorescent material can be coupled to the surface of the beads or incorporated into the bead. For instance, magnetically responsive beads can be dyed using techniques known to those in the art, such as those described in U.S. Pat. No. 6,514,295. Briefly, an organic solvent is used to swell the bead, allowing fluorescent dye to enter it.

By varying the concentrations of dyes (various signal intensities) and using different dye combinations (different dye emissions) that are incorporated into the beads, a plurality of distinguishable bead sets can be generated (see, e.g., U.S. Pat. No. 5,981,180). For instance, a set of beads can be generated by doping the beads with different ratio of 2 or more different dyes, e.g., 2, 3, 4, 5, 6, 7, 8, 9, 10 or more different dyes. A set of beads can also be generated using 2 different dyes at different ratios, such that a first dye is incorporated into the bead at a plurality of unique dye concentrations and a second dye is incorporated into the bead at a plurality of unique dye concentrations. For instance, the set of bead can include an array of beads each with a unique dye profile that is established by doping each bead with a different dye combination of two different fluorescent dyes.

### III. PREPARATION OF BLOCKING AGENTS

The blocking agent can be formed by coating the binding agent to the surface of the bead. Without intending to limit the method of preparation, in some embodiments, an amine-based conjugation reaction can be performed to couple the agent with a primary amine to a carboxylated bead. Firstly, an active ester is formed by a reaction between the carboxylated bead and 1-cyclohexyl-3-(2-morpholinoethyl)carbodiimide (CMC) and 1-hydroxybensotriazole (HOBt). Next, the binding agent is covalently coupled to the bead surface via the reactive ester. For binding agents that do not carry primary amines, the bead can be derivative to generate an appropriate function group prior to attaching the binding agent. For example, a two-step carbodiimide reaction can be performed as follows: 1) the carboxyl group on the surface of the bead is activated with a carbodiimide such as EDAC (1-ethyl-3-[3-dimethylaminopropyl]carbodiimide hydrochloride) which forms an active O-acylisourea intermediate, and 2) the active ester reacts with primary amines of the binding agent to form a covalent bond.

The bead can have more than one type of functional reactive group, e.g., 2, 3, 4, 5 or more different functional groups, on its surface. In some instances, the bead can have at least one functional reactive group.

As noted above, in some cases, at least two separate and different binding agents can be conjugated to a single bead. In some embodiments, the binding agents are on the bead at different ratios. In some embodiments, the ratio of the binding agents ranges from about 1:1.5 to 1:10, e.g., 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, or 1:10. For instance, the first binding agent is covalently coupled at a specific amount and the second binding agent is affixed at a higher or lower amount, but not the same amount.

### IV. METHOD OF USING BLOCKING AGENTS

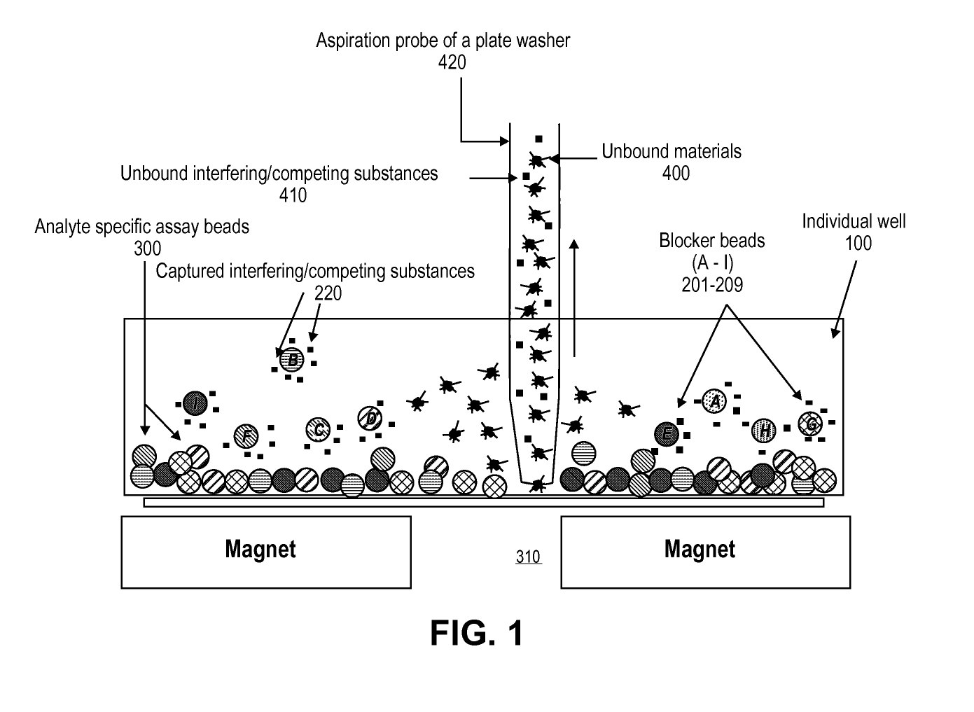

FIG. 1 illustrates a use of blocking reagents as described herein. A plurality of blocking agents (e.g., blocker beads) can be admixed together with one or a plurality of affinity agents (e.g., assay beads). Depending upon when the blocking agent is applied, the blocking agent can capture interfering molecules present in the sample, the assay mixture (the sample and the affinity agent(s)), or both. The presence of the blocking agent enhances the specificity of the assay by reducing the effect of the interfering molecules in the sample on the binding of the affinity agent to the target analyte.

If more than one type of blocking agent is needed, the different blocking agents can be used at an equal amount (e.g., equal ratio) or at a different amount (e.g., different ratio). For example, in some instances, the same amount of the first blocking agent and the second blocking agent are added to the sample, the affinity agent or the assay mixture. In other instances, the first blocking agent is added at a ratio of at least 2 times or more, e.g., 2, 3, 4, 5, 6, 7, 8, 9, 10 or more, than the second blocking agent. The amount of blocking agent(s) needed in the method described herein is dependent on the sample, the assay reagents, and assay conditions. Therefore, the selection of the blocking agent(s) as well as the necessary amount to be used can be determined experimentally.

FIG. 1 depicts an individual assay well 100 that contains blocking agent 201-209 (blocker beads A-I), a complex between a blocker bead B and interfering molecules 220, analyte specific assay beads 300, a magnetic material generating a magnetic field 310, unbound material of the sample 400, unbound interfering and/or competing molecules 410, and an aspiration probe of a plate washer 420. As depicted, the blocking reagent and affinity reagent are distinguishable by fluorescence (indicated by different color beads), wherein the blocking reagent is either not fluorescent or emits fluorescence at a wavelength that minimally overlaps with that of the affinity reagent. Likewise, the presence or level of the target molecule(s) can be determined from the fluorescence of the affinity reagent(s) (not shown) and the reporter that labels the detection antibody (not shown).

In some embodiments, blocking agents can be used with an antibody sandwich immunoassay. For instances, blocking agents can be used in an assay (e.g., ELISA), wherein the capture antibody serves to capture the target molecule of interest and is coupled to a solid phase, e.g., well, bead, etc.

In some embodiments, the blocking agent can be added to the sample directly. The blocking agent(s) can be selected according to the sample type(s) used in the assay. For instances, a blocking agent comprising an antibody against HAMA and a blocking agent comprising an antibody against rheumatoid factor (RF) can be used to remove interfering molecules such as HAMA and RF present in a human serum sample.

In some embodiments, the blocking agent is added to the assay mixture. In some instances, the blocking agent and the affinity agent are added simultaneously to the sample. In other instances, the blocking agent and affinity agent are added sequentially to the sample. In yet other instances, the blocking agent is added to the assay mixture containing the sample and the affinity agent.

In some embodiments, the blocking agent is incubated at a temperature and for a duration of time such that a non-specific binding complex comprising the blocking agent and one or more interfering molecules is formed. In some instances, the blocking agent binds to different interfering molecules.

Any relevant method can be used to separate the non-specific binding complex from the sample. In some embodiments, the blocking agent and the non-specific binding complex are separated from the sample by centrifugation. In other embodiments, if the blocking agent is responsive to a magnetic field, a magnetic field is applied to the non-specific binding complex, and then the sample or assay mixture is separated from the complex.

In some embodiments, the non-specific binding complex is not removed from the assay mixture prior to the detection and/or quantitation of the affinity agent bound to the target analyte.

Typically, the fluorescent dyes of the blocking agent and the affinity agent are selected based on their ability to emit light in the wavelength of the detection window of the system. Typically, the detection windows are chosen to be spaced apart by a number of wavelengths, and the dyes are chosen in order to minimize the overlap of the dye's fluorescent signal with the detection windows.

### V. IMMUNOASSAYS

Examples of immunoassays to which the blocking agents described herein can be applied include, enzyme linked immunoabsorbent assay (ELISA), fluorescent immunosorbent assay (FIA), immunohistochemistry, free or ambient analyte immunoassays, microsphere-based immunoassays, chemical linked immunosorbent assay (CLIA), radio-immuno assay (RIA), flow cytometry (e.g., fluorescence activated cell sorting or FACS), Western blot, Southern blot, and immunoblotting. Additional applicable immunotechniques include competitive and non-competitive assay systems, e.g., “sandwich” immunoassays, immunoprecipitation assays, precipitin reactions, immunodiffusion assays, immunoradiometric assays, fluorescent immunoassays, etc. Immunoassays can be multiplex, with multiple simultaneous or sequential assays, or carried out automatically, e.g., using Bio-Plex® or similar systems. For a review of immunoassays for which the presently described blocking reagents can be used, see, e.g., The Immunoassay Handbook, David Wild, 3^{rd} ed., Stockton Press, New York, 2005; Ausubel et al, eds, 1994, Current Protocols in Molecular Biology, Vol. 1, John Wiley & Sons, Inc., New York.

An exemplary flow cytometry-based multiplex assay (e.g., Bio-Rad Bio-Plex assay, Luminex xMAP assay, etc.) allows multiple analytes to be assayed simultaneously in a single sample. The assay utilizes microspheres (e.g., microparticles or beads) with a diameter of about 5-6 μm that are internally labeled with two fluorescent dyes. As the microspheres passes through the flow cell, it is interrogated by two lasers. One laser identifies the microsphere on the basis of the ratio of the two fluorophores contained within the microsphere, while the other laser quantitates the amount of analyte bound to the microsphere on the basis of the intensity of the reporter fluorescence. The surface of each microsphere can contain multiple reactive groups that function as sites for covalent biomolecule (e.g., ligand or antibody) attachment.

### VI. KITS

In some embodiments, the kit comprises more than one (e.g., at least two) blocking agent, as described herein. In some embodiments, the kit comprises one or more (e.g., at least two) affinity agent(s). In some aspects, the affinity agent comprises an antibody or a fragment thereof.

The blocking agent can be a bead linked to a binding agent. In some instances, the bead of the blocking agent is coupled to one or more binding agents. Example of the binding agent include, but are not limited to, bovine serum albumin (BSA), protein L, collagen, PEG4000/6000, animal serum, a murine based IgG aggregate, and an antibody derived from goat, mouse, rabbit or sheep that recognizes a HAGA, HAMA, HARA, HASA, or rheumatoid factor.

In some embodiments, the bead of the blocking agent comprises a fluorescent dye distinguishable from other fluorescent dyes used in the affinity assay. In other embodiments, the bead does not generate a fluorescent signal. In some embodiments, the beads of the kit (e.g., beads of the blocking agent and beads of the affinity agent) comprise a set of fluorescent dyes that are distinguishable from the other fluorescent dyes of the kit. The bead of the blocking agent can be non-magnetic. The bead can be responsive to a magnetic field (e.g., superparamagnetic).

The affinity agent can be linked to a bead, wherein the bead is responsive to a magnetic field (e.g., superparamagnetic). In some embodiments, the bead of the affinity agent comprises fluorescent dye distinguishable from other fluorescent dyes used in the assay. The bead of the affinity agent can be non-magnetic. The bead can be responsive to a magnetic field (e.g., superparamagnetic). In some aspects, the affinity agent comprises an antibody or a fragment thereof. The affinity agent is selected to bind a specific target analyte of the assay.

In some embodiments, the kit includes supplies and reagents for carrying out an immunoassay, such as ELISA plates, buffer stock solutions, standards and/or controls, magnets, etc.

The kit will also typically include instructions for use, or direction to an outside source of instruction such as a website.

### VII. EXAMPLES

### Example 1

### Using Blocking Agents to Remove Interfering Molecules from a Sample Prior to Affinity Assay

This example describes a method of preparing samples using blocking agents. A sample such as serum is incubated with blocking agents selected according to their affinity for specific interfering molecules known to be present in the sample. The admixture of the sample and the blocking agents are incubated under conditions such that the blocking agents form complexes with the interfering molecules. The complex and the unbound blocking agents are separated from the sample by centrifugation (or by any known methods in the art). The sample which is now free or substantially free of interfering molecules is then collected.

### Example 2

### Using Blocking Agents in Magnetic Bead-Based Affinity Assay

This example illustrates an exemplary embodiment of the method provided herein (see, FIG. 1). In particular, the example describes a protocol for a multiplex magnetic bead-based affinity assay (e.g., Bio-Rad Bio-Plex system) that can be used to measure the level of different target analytes in a sample.

The components of the assay include a sample (containing the target analytes and interfering molecules), affinity agents, blocking agents, a magnet, a plate washer, and a fluorescence detector. The affinity agents (analyte specific assay beads 300) are a series of fluorescent-coded magnetic beads, each of which is coupled to a unique antibody specific for a particular target analyte (molecule). The blocking agents (blocker beads 201-209) are coupled to different binding agents (e.g., binding agent A-I) that can bind one or more interfering molecules present in the assay well (individual well 100).

A test sample, standards and quality control samples are obtained. Standards are used to generate a standard curve. In particular, a standard dilution series of standards is made to produce an eight-point standard curve with a four-fold dilution between each point.

The test sample is added to the assay well. A series of pre-selected blocker agents is added to the sample, followed by the addition of a panel of affinity agents. The admixture is incubated for 30 minutes at room temperature with shaking at 850 rpm to allow the blocking agents to capture interfering molecules 220 present in the sample and the affinity agents to bind to their target analytes. A magnet 310 is placed at the bottom of the well such that a magnetic field is applied to the affinity agents. The well is washed three times with a wash buffer. A plate washer with an aspiration probe is used to remove unbound interfering molecules 410 (interfering molecules not are not bound to the blocking agents) and other unbound materials 400. Next, a biotin conjugated detection antibody is added to the well and the admixture is incubated for 30 minutes at room temperature with shaking at 850 rpm. The well is then washed three times using wash buffer and a plate washer. Next, streptavidin-PE is added to the well and the admixture is incubated for 10 minutes at room temperature with shaking for 850 rpm. The well is once again washed three times with wash buffer using a plate washer. The well is removed from the magnetic field and the contents in the well (e.g., affinity agents) are resuspended in the assay buffer. The fluorescent signal from the affinity agents are acquired using a flow cytometer with two lasers and associated optics or a fluorescent plate imager with a LED/CCD camera. The fluorescent signal from the affinity agent correlates to the level of the specific target analyte present in the sample.

### Example 3

### Using Blocking Agents in an Affinity Assay

This example illustrates an exemplary embodiment of the method for removing interfering molecules from an affinity assay mixture, wherein the mixture is of a sample and an affinity agent. In this example the affinity agent is not bound to a bead.

Blocking agents are admixed with an affinity assay mixture containing a sample and an affinity agent and incubated for about 30-60 minutes at room temperature to allow the interfering molecules of the assay mixture to form a complex with the blocking agents. Next, the complex is removed from the assay mixture by centrifugation. Detection of the affinity agent bound to the target analyte is performed according to standard methods known in the art.

### Example 4

### Using Blocking Agents with Affinity Agent Solutions

This example illustrates an exemplary embodiment of the method for removing interfering molecules from an affinity agent solution such as a reagent containing an antibody or a fragment thereof by using magnetic blocking agents.

Blocking agents (e.g., a set of beads linked to different binding agents) are added to an affinity agent solution that contains one or more molecule that interferes with the binding of the affinity agent to its target analyte (molecule). The mixture is incubated for about 30 minutes are room temperature to allow the blocking agent to form a complex with the interfering molecule(s). The complex is then separated from the affinity agent solution by applying a magnetic field to the mixture and the processed affinity agent solution is collected and used in standard affinity assays (e.g., immunoassays).

It is understood that the examples and embodiments described herein are for illustrative purposes only and that various modifications or changes in light thereof will be suggested to persons skilled in the art and are to be included within the spirit and purview of this application and scope of the appended claims. All patents, patent applications, internet sources, and other published reference materials cited in this specification are incorporated herein by reference in their entireties. Any discrepancy between any reference material cited herein or any prior art in general and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. A method of blocking an interfering molecule in an affinity assay mixture comprising a sample, the method comprising
(a) contacting a first blocking agent to the sample, wherein the first blocking agent comprises a first bead linked to a first binding agent, thereby forming a non-specific binding complex between the first binding agent and one or more molecule in the sample that would otherwise interfere with forming an affinity complex between an affinity agent and a target molecule, if present, in the sample; and
(b) contacting a second blocking agent to the sample, wherein the second blocking agent comprises a second bead linked to a second binding agent that is different from the first binding agent, thereby forming a non-specific binding complex between the second binding agent and one or more molecule in the sample that would otherwise interfere with forming an affinity complex between the affinity agent and the target molecule, if present, in the sample; thereby blocking the interfering molecule in the assay mixture.

2. The method of claim 1, wherein the first binding agent and the second binding agent bind to different interfering molecules.

3. The method of claim 1, wherein the bead of the blocking agent is a non-magnetic bead.

4. The method of claim 1, wherein the bead of the blocking agent is a bead that is responsive to a magnetic field.

5. The method of claim 1, wherein the binding agent is selected from the group consisting of BSA, protein L, collagen, PEG4000/6000, animal serum, a murine based IgG aggregate, and an antibody derived from goat, mouse, rabbit or sheep that recognizes a HAGA, HAMA, HARA, HASA, or rheumatoid factor.

6. The method of claim 1, wherein the first blocking agent and/or second blocking agent comprises more than one binding agent.

7. The method of claim 1, wherein the first blocking agent and the second blocking agent are at a substantially equal ratio.

8. The method of claim 1, wherein the first blocking agent is at a ratio of at least about 2 times or more than the second blocking agent.

9. The method of claim 1, further comprising removing the non-specific binding complex from the other components of the assay mixture.

10. The method of claim 1, further comprising contacting the affinity agent to the sample; and detecting the presence or quantity of the affinity complex.

11. A plurality of blocking agents comprising a first bead linked to a first binding agent and a second bead linked to a second binding agent that is different that the first binding agent, wherein the binding agent is selected from the group consisting of BSA, protein L, collagen, PEG4000/6000, animal serum, a murine based IgG aggregate, and an antibody derived from goat, mouse, rabbit or sheep that recognizes a HAGA, HAMA, HARA, HASA, or rheumatoid factor.

12. The plurality of blocking agents of claim 11, wherein the bead is a non-magnetic bead.

13. The plurality of blocking agents of claim 11, wherein the bead is responsive to a magnetic field.

14. The plurality of blocking agents of claim 11, wherein the first bead and/or second bead is linked to at least two different binding agents.

15. A kit comprising a plurality of blocking agents and an affinity agent specific for a target molecule, wherein the plurality of blocking agents comprises a first blocking agent comprising a bead linked to a first binding agent that forms a non-specific binding complex to one or more interfering molecule in a sample and a second blocking agent comprising a bead linked to a second binding agent that is different than the first binding agent and forms non-specific binding complex to one or more interfering molecule in a sample.

16. The kit of claim 15, wherein the bead is responsive to a magnetic field.

17. The kit of claim 15, wherein the binding agent is selected from the group consisting of BSA, protein L, collagen, PEG4000/6000, animal serum, a murine based IgG aggregate, and an antibody derived from goat, mouse, rabbit or sheep that recognizes a HAGA, HAMA, HARA, HASA, or rheumatoid factor.

18. The kit of claim 15, wherein the bead of the first blocking agent and/or second blocking agent is linked to at least two different binding agents.

19. The kit of claim Error! Reference source not found.15, wherein the affinity agent further comprises bead.

20. The kit of claim 19, wherein the bead comprises a fluorescent dye that is distinguishable from other components of the kit.
